(19) United States
(12) Patent Application Publication
(10) Pub. No.: US 2009/0030469 A1
(43) Pub. Date: Jan. 29, 2009

(54) Cardiac Resynchronization Therapy Systems and Methods

(76) Inventor: Gideon Meiry, Ashrat (IL)

Correspondence Address: THE NATH LAW GROUP, 112 South West Street, Alexandria, VA, 22314, US

(21) Appl. No.: 11/990,306

(22) PCT Filed: Aug. 10, 2006
(86) PCT No.: PCT/IL2006/000932

§ 371 Date: Jun. 19, 2008

Related U.S. Application Data
(60) Provisional application No. 60/707,155, filed on Aug. 11, 2005.

(51) Int. Cl. A61N 1/368; A61N 1/39; A61N 1/05

(52) U.S. Cl. 607/4; 607/126; 606/129; 607/9

(57) ABSTRACT

Systems and methods for cardiac resynchronization therapy include a right side component and a left side component, wherein the right side component includes at least one ventricular lead and the left side component includes at least one epicardial electrode. Epicardial electrodes are insertable via a minimally invasive procedure, and are positionable via imaging techniques.

### FIELD AND BACKGROUND OF THE INVENTION

The present invention is directed to cardiac resynchronization therapy (CRT) in heart failure patients. More specifically, the present invention is directed to positioning left ventricle electrodes at targeted locations of the epicardium.

Congestive Heart Failure (HF) is a common clinical syndrome resulting from heart damage, wherein the metabolic demands of the body are not met by the output of the heart. Many heart failure patients have an Inter-Ventricular Conduction Delay (IVCD), typically manifested as Left Bundle Branch Block (LBBB). In a normal cardiac cycle, the left and right atria contract simultaneously, followed by the left and right ventricles. IVCD results in abnormal electrical depolarization of the ventricles, causing the two ventricles to beat asynchronously, which may lead to increased mortality in heart failure patients.

A recent approach aimed at treating IVCD is termed Cardiac Resynchronization Therapy (CRT), or biventricular pacing. In principle, a CRT device is similar to a pacemaker. As in a dual chamber pacemaker, a pulse generator is implanted under the skin of the upper chest, and two leads are fixed transvenously to the endocardial wall, generally one in the right atrium and one in the right ventricle. In CRT, an additional lead is implanted transvenously via the coronary sinus and positioned in a cardiac vein to sense and pace the left ventricle.

Implantation of the additional lead requires an accessory kit including a steerable catheter left-heart lead delivery system. Contrast media injection for improved visualization of the cardiac venous anatomy is needed during implantation. The left-heart electrode is advanced through the coronary sinus and into the target branch vein. CRT is delivered as tiny electrical pulses to both the right and left ventricles, synchronizing the activation of the ventricles, and thus reducing mitral regurgitation and improving left ventricle filling.

However, pacing the left ventricle remains problematic. Studies have revealed that it is more difficult to fix a lead into the left ventricular cavity, although it is easy to do so in the right ventricle. This is mainly due to the higher blood pressure in the left ventricle (˜7 times larger that of the right ventricle). Moreover, an electrode in the left ventricle may serve as a nidus for clot formation and systemic emboli. Accordingly, as described above, today's CRT devices sense and pace the left ventricle by a third lead which is implanted via the coronary sinus and positioned in a cardiac vein, coming from the left ventricular wall.

An example of a pacing system used for CRT is disclosed in U.S. Pat. No. 6,754,530 to Bakels et al. The system and method disclosed therein includes the use of impedance sensors for determining optimum pacing parameters, e.g., for pacing the left ventricle so that left heart output is maximized. Pacing of the left ventricle is accomplished by positioning a lead via the coronary sinus into a cardiac vein such as the middle or great cardiac vein, such that a distal electrode is in a position for pacing of the left ventricle.

Another example of a pacing system used for CRT is disclosed in U.S. Pat. No. 6,701,186 to Spinelli et al. The device includes a right atrium electrode and one or more electrodes located at one or more ventricular walls. As in other CRT devices, the left ventricle pacing conductor which leads to the left ventricle electrode is positioned at a location such as within a lateral branch of the coronary sinus vein spanning the left ventricle.

Thus, known devices for CRT include a left ventricle lead and electrode positioned in a cardiac vein which is within the left ventricular wall. This technology does not facilitate much flexibility as only a few large veins emerge from the left side of the heart. Moreover, advancing the electrode through such veins is limited since they become narrower further away from the coronary sinus. In addition, not all veins are in good condition at progressive stages of a failing heart, and inability to canulate the coronary sinus or inability to obtain a stable pacing site occurs in more then 10% of the CRT population. Physicians are often left with a possibility of placing the left ventricle electrode wherever they can, rather than in the desired location. Mismatches between the site of maximal delay and the site of electrode positioning are thought to be a primary reason for failure of left ventricle synchronization. Moreover, although it is probable that more than one site of conduction delay would benefit from pacing, today's CRT devices include only one electrode for pacing the left ventricle due to the complexity of placement and fixation.

Furthermore, depending on circulating neurohormones, mechanical load, and possibly genetic factors, the heart of each individual patient differs in size, shape, stage of the disease, and performance, i.e., each patient's heart has its own hallmarks, resulting in unique individual activity. Current CRT techniques do not account for differences in heart function in individuals.

There is thus a widely recognized need for, and it would be highly advantageous to have, a CRT method and apparatus which is devoid of the above limitations.

### SUMMARY OF THE INVENTION

According to one aspect of the present invention there is provided a system for cardiac resynchronization therapy of a heart. The system includes a right side component including at least one right side lead positionable inside the heart and configured to provide sensing signals from sensed electrical activity of the heart and to receive pacing signals for pacing electrical activity of the heart, a left side component including at least one epicardial electrode positionable on an epicardial surface of the heart and configured to provide sensing signals from sensed electrical activity of the heart and to receive pacing signals for pacing electrical activity of the heart, and at least one pulse generator for receiving from the right side component and the left side component the sensing signals provided from sensed electrical activity and for sending the pacing signals to the right side component and the left side component.

According to another aspect of the present invention there is provided a kit for providing enhanced cardiac resynchronization therapy to an existing single-side pacing system or double side cardiac resynchronization therapy system, the existing system having a right side component and a first pulse generator in communication with the right side component. The kit includes a left side component having at least one epicardial electrode positionable on an epicardial surface of the heart and configured to provide sensing signals from sensed electrical activity of the heart and to receive pacing signals for pacing electrical activity of the heart, a second pulse generator for receiving from the left side component the sensing signals provided from sensed electrical activity and for sending the pacing signals to the left side component, and a telemetry module in communication with the second pulse generator, for sending sensing signals and pacing signals to the first pulse generator.

According to yet another aspect of the present invention there is provided a method for positioning a system for cardiac resynchronization therapy. The method includes imaging a cardiac region of the body during a regular cardiac cycle, determining at least one location having a conduction disturbance based on the imaging, inserting a steering instrument into the cardiac region, the steering instrument having at least one epicardial electrode stored therein, temporarily placing the at least one epicardial electrode on an epicardial surface at the at least one location, testing the at least one epicardial electrode for efficacy, determining a placement location based on the imaging and the testing, and fixing the at least one epicardial electrode to an epicardial surface at the placement location.

According to yet another aspect of the invention there is provided an epicardial electrode for temporary attachment to an epicardium wall. The electrode includes a contact portion configured to be in electrical contact with an epicardium of a heart, and a suction portion, where the suction portion includes a flexible band surrounding the contact portion, at least one suction valve on the contact portion, and a vacuum tube connecting the at least one suction valve to a suction pump.

According to yet another aspect of the invention, there is provided a delivery tool for minimally invasive placement of epicardial electrodes. The tool includes a distal end having a holder for holding of at least one of the epicardial electrodes, a proximal end having a handle, and a body connecting the distal and proximal ends, wherein the body includes at least one articulating element, wherein the handle includes controls for controlling the at least one articulating element and the at least one epicardial electrode.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a partially cut anterior view of a heart with a system for CRT in accordance with a preferred embodiment of the present invention;

FIG. 2 is a partially cut anterior view of a heart with a system for CRT in accordance with an alternative embodiment of the present invention;

FIG. 3 is a perspective illustration of an epicardial electrode in accordance with one embodiment of the present invention;

FIG. 4 is a perspective illustration of a delivery system in accordance with one embodiment of the present invention

FIG. 5 is a schematic illustration of a positioning system for positioning of epicardial electrodes on the epicardial surface of the heart;

FIG. 6 is a flow chart diagram of a method of positioning of epicardial electrodes in accordance with a preferred embodiment of the present invention;

FIG. 7 is a schematic illustration of an overview of a method of pacing using the system of the present invention;

FIG. 8 is a schematic illustration of an overview of an alternative method of pacing using the system of the present invention;

FIG. 9 is a block diagram illustration of the components of pulse generator; and

FIG. 10 is a flow chart illustration of a method of pacing with a defibrillator option, in accordance with one embodiment of the present invention.

### DESCRIPTION OF THE PREFERRED EMBODIMENTS

The present invention is of a method and system which can be used for cardiac resynchronization therapy (CRT).

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

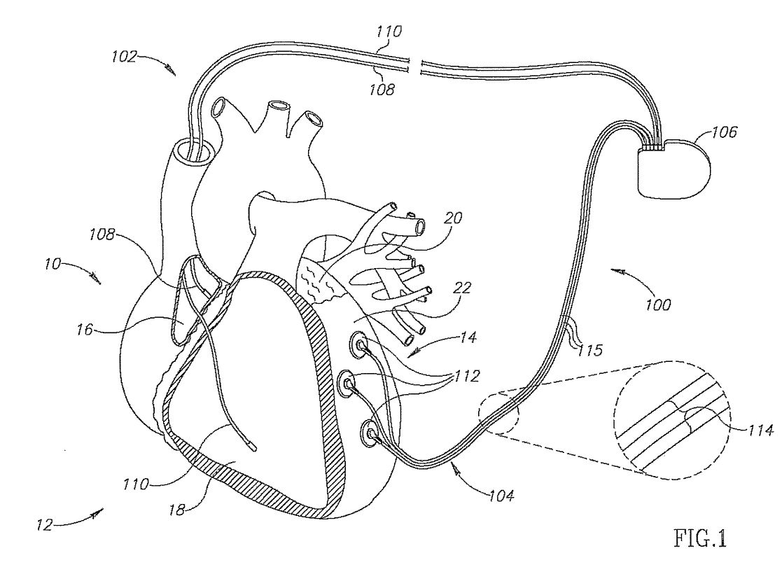

Reference is now made to FIG. 1, which is a partially cut anterior view of a heart 10 with a system 100 for CRT in accordance with a preferred embodiment of the present invention. Heart 10 has a right side 12 and a left side 14. Right side 12 includes a right atrium 16 and a right ventricle 18, shown in cut-away view so that the interiors of both right atrium 16 and right ventricle 18 are visible. Left side includes a left atrium 20 and a left ventricle 22, shown in perspective view so that the exterior surfaces of both left atrium 20 and left ventricle 22 are visible.

System 100 includes a right side component 102, a left side component 104, and a pulse generator 106. Right side component 102 includes two leads, which are preferably soft insulated wires, and are positionable inside heart 10: right atrium lead 108 leading into right atrium 16, and right ventricle lead 110 leading into right ventricle 18. Tips of leads 108 and 110 are fixed to the myocardium wall. Each of the leads of right side component 102 is in electronic communication with pulse generator 106. In a preferred embodiment, the communication between leads of right side component 102 and pulse generator 106 is via conducting wires. In an alternative embodiment, the communication between leads of right side component 102 and pulse generator 106 is wireless. Left side component 104 includes at least one epicardial electrode 112 for placement on the epicardium of left ventricle 22. In a preferred embodiment, several epicardial electrodes 112 are used in different locations on the epicardium, the positions of which are determined in a manner which is described more fully herein below. The number of epicardial electrodes 112 can be any number, from one to an unlimited number of electrodes, and depends on the particular needs of the individual. Leads from epicardial electrodes 112 are in electronic communication with pulse generator 106. In a preferred embodiment, the communication between leads from epicardial electrodes 112 and pulse generator 106 is via conducting wires. In an alternative embodiment, the communication between leads from epicardial electrodes 112 and pulse generator 106 is wireless. In one embodiment, leads 115 from several epicardial electrodes 112 are joined in a common bundle 114 of insulated connecting wires, which then leads directly to pulse generator 106. In a preferred embodiment, pulse generator 106 is positioned in the chest wall, as is commonly known in the art.

As stated above, right side component 102 includes leads which are positionable inside right atrium 16 and right ventricle 18. Each of these leads includes an electrode at a distal end thereof. Such leads and electrodes are well known in the art and may be obtained, for example, from Medtronic Inc, CapSureFix Novus 5076 Steroid-Eluting, Screw-In Pacing Lead. Left side component 104 includes at least one epicardial electrode 112. Epicardial electrode 112 is in principle similar to commercially available epicardial lead electrodes such as Medtronic Inc, CapSure Epi 4968 Steroid-Eluting, Epicardial Pacing Leads, or EN-Path medical Inc, MyoPore® epicardial leads, capable of both sensing and pacing. However, known epicardial electrodes are not capable of temporary attachment. As such, epicardial electrodes 112 of the present invention are adapted for use in the system and method described herein. Adaptations include, for example, inclusion of means for temporary attachment to the epicardium wall, and markers for monitoring and mapping of the electrode position during implantation, as described further herein below. Pulse generator 106 is a modified pulse generator as found in typical CRT pacemaking systems such as, for example, Medtronic Inc, InSync Sentry™ CRT-D Device. Modifications may include, for example, adaptations to enable sensing and pacing of multiple electrodes. In one embodiment, pulse generator 106 includes several channels. In a preferred embodiment, pulse generator 106 is implanted in the chest wall by a minimally invasive surgical procedure, as is commonly known in the art.

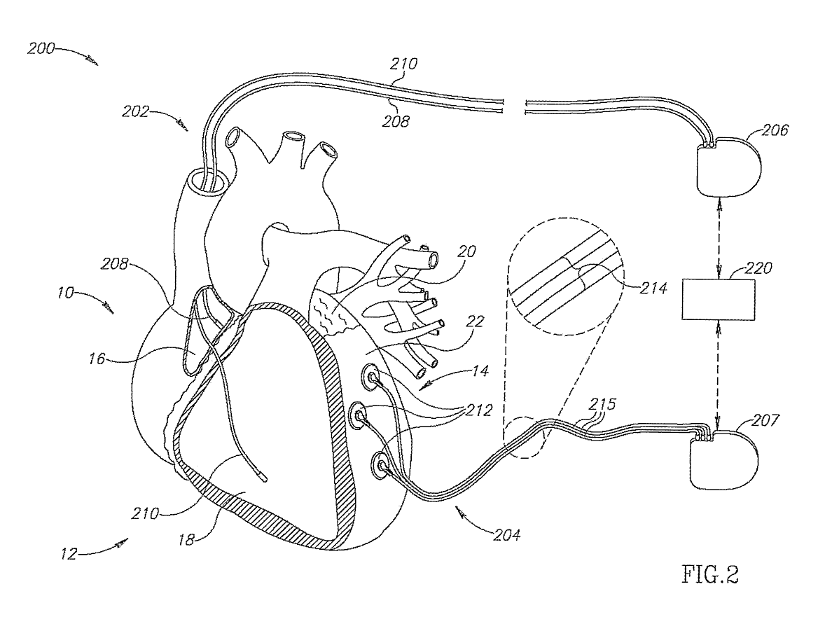

Reference is now made to FIG. 2, which is a partially cut anterior view of heart 10 with a system 200 for CRT in accordance with an alternative embodiment of the present invention. Heart 10 is shown with right side 12 having a cut-away view, and left side 14 having a perspective view so that the interior portion of right atrium 16 and right ventricle 18 are visible, while an exterior surface of left atrium 20 and left ventricle 22 are visible.

System 200 includes a right side component 202, a left side component 204, a right side pulse generator 206 and a left side pulse generator 207. Right side component 202 includes two leads which are positionable inside heart 10: right atrium lead 208 leading into right atrium 16, and right ventricle lead 210 leading into right ventricle 18. Tips of leads 208 and 210 are fixed to the myocardium wall. Each of the leads of right side component 202 is in electronic communication with right side pulse generator 106. In a preferred embodiment, the communication between leads of right side component 202 and pulse generator 206 is via conducting wires. In an alternative embodiment, the communication between leads of right side component 202 and pulse generator 206 is wireless. Left side component 204 includes at least one epicardial electrode 212 for placement on the epicardium of left ventricle 22. In a preferred embodiment, several epicardial electrodes 212 are placed in different locations on the epicardium, the positions of which are pre-determined in a manner which is described more fully hereinbelow. The number of epicardial electrodes 212 can be any number, from one to an unlimited number of electrodes, and depends on the particular needs of the individual. Leads from epicardial electrodes 212 are in electronic communication with left side pulse generator 207. In a preferred embodiment, the communication between leads from epicardial electrodes 212 and pulse generator 207 is via conducting wires. In an alternative embodiment, the communication between leads from epicardial electrodes 212 and pulse generator 207 is wireless. In one embodiment, leads 215 from several epicardial electrodes 212 are joined in a common bundle 214 of insulated connecting wires, which then leads directly to left side pulse generator 207.

As stated above, right side component 202 includes leads which are positionable inside right atrium 16 and right ventricle 18. Each of these leads includes an electrode at a distal end thereof. Such leads and electrodes are well known in the art and may be obtained, for example, from Medtronic Inc, CapSureFix Novus 5076 Steroid-Eluting, Screw-In Pacing Lead. Left side component 204 includes at least one epicardial electrode 212. Epicardial electrode 212 is in principle similar to commercially available epicardial lead electrodes such as Medtronic Inc, CapSure Epi 4968 Steroid-Eluting, Epicardial Pacing Leads, or EN-Path medical Inc, MyoPore® epicardial leads, capable of both sensing and pacing. However, known epicardial electrodes are not capable of temporary attachment. As such, epicardial electrodes 112 of the present invention are adapted for use in the system and method described herein. Adaptations include, for example, inclusion of means for temporary attachment to the epicardium wall, and markers for monitoring and mapping of the electrode position during implantation, as described further herein below. Right side pulse generator 206 is any commercially available pulse generator, such as Medtronic Inc, Intrinsic™ Dual Chamber ICD with MVP™ Mode (Managed Ventricular Pacing). Left side pulse generator 207 is a modified pulse generator which can be based on a commercial CRT pulse generator such as Medtronic Inc, InSync Sentry™ CRT-D Device. Modifications may include adaptations to enable sensing and pacing of multiple electrodes. In one embodiment, pulse generator 106 includes several channels.

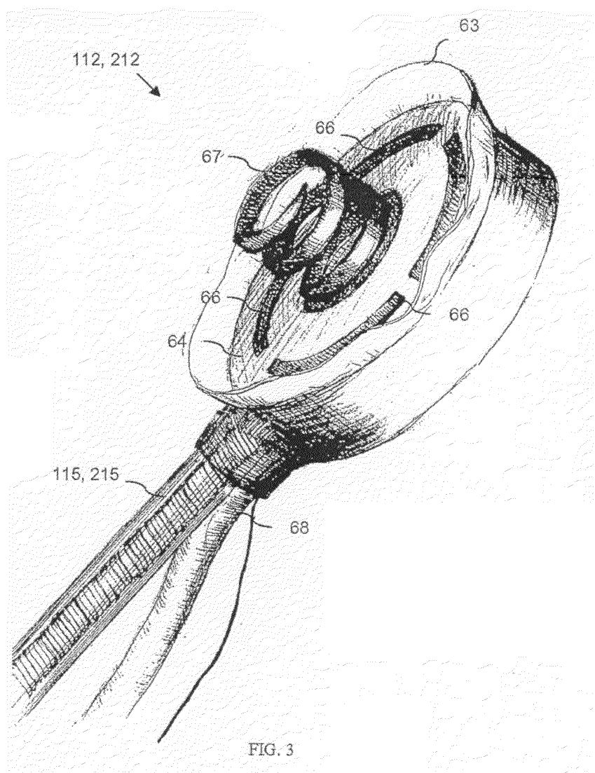

Reference is now made to FIG. 3, which is a perspective illustration of an epicardial electrode 112 or 212 in accordance with one embodiment of the present invention. Epicardial electrode 112, 212, includes a conductive portion and a suction portion. The conductive portion is comprised of a conducting material as is commonly used for electrodes, and includes a contact surface 64 and electrical lead 115, 215. The suction portion includes a flexible band 63, at least one suction valve 66, and a vacuum tube 68. Flexible band 63 is comprised of a flexible elastic material, such as silicone, for creating suction between contact surface 64 of epicardial electrode 112, 212 and the epicardium of the heart. Flexible band 63 surrounds and protrudes out from contact surface 64. At least one suction valve 66 is placed within a surface of epicardial electrode 112, 212, and is connected to vacuum tube 68. In one embodiment, several suction valves 66 are present, each of which is connected to vacuum tube 68. Vacuum tube 68, which can be, for example, a miniature flexible tube made from silicone, is positioned alongside electrode lead 115 or 215, and is connected to at least one suction valve 66 on contact surface 64 of epicardial electrode 112, 212. A distal side of vacuum tube 68 is connected to a suction pump. Thus, suction portion fits directly onto the epicardial surface of heart 10 when electrode 112, 212 is attached thereto, thus creating a sealed space between the electrode surface and the epicardium wall. If suction is turned on, air is sucked out from the sealed space, generating a vacuum and causing electrode 112, 212 to be attached to the epicardium wall until suction is turned off. The vacuum tube can be permanently or temporarily integrated with electrode lead 115 or 215. In one embodiment, electrode 115, 215 further includes a fixation element 67 for permanent fixation to the epicardium wall. Fixation element may be, for example, a metal screw or any other means for fixing an electrode to the epicardium wall, as is commonly known in the art.

In a preferred embodiment, right side pulse generator 206 is implanted in the chest wall by a minimally invasive surgical procedure, and left side pulse generator 207 is separately implanted in a different location, such as under the abdominal wall, also by minimally invasive surgery. In one embodiment, right side pulse generator 206 and left side pulse generator 207 work in coordination indirectly by individually sensing electrical activity of the heart and pacing it accordingly. In an alternative embodiment, right side pulse generator 206 and left side pulse generator 207 work in direct coordination, wherein communication between right side pulse generator 206 and left side pulse generator 207 is accomplished by using a telemetry module 220. Telemetry module 220 is included in both right side pulse generator 206 and left side pulse generator 207, and enables “cross-talk” between left and right side pulse generators 206, 207. That is, telemetry module 220 enables both pulse generators to transmit and/or receive data from the alternate pulse generator, allowing them to perform in synchronicity and as a single unit.

Delivery systems are well known in the art and may be obtained, for example, from EN-Path Medical Inc, implant tool (FasTac®). However currently available delivery systems and procedures are limited in their steering and flexibility, and may not be adequate for accurate placement of electrodes at specific locations on the epicardium. In addition, each electrode requires its own delivery catheter and each placement of electrode requires a new cut in the patient's chest wall. Moreover none of the currently used delivery systems is capable of temporarily placing an epicardial electrode without wounding the myocardium.

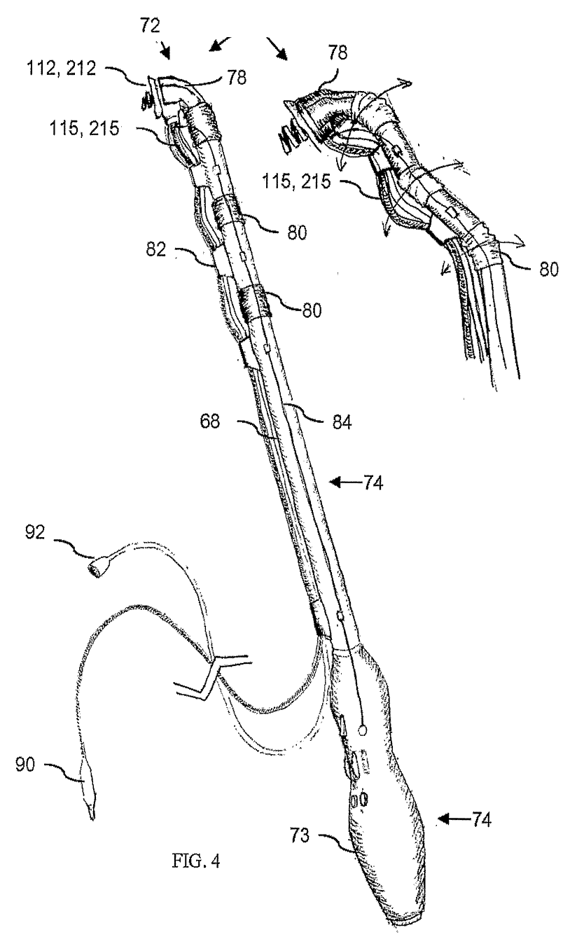

Reference is now made to FIG. 4, which is a perspective illustration of a delivery system in accordance with one embodiment of the present invention. A delivery tool 70 has a distal end 72, a proximal end 74, and a body 76 connecting distal end 72 to proximal end 74. Distal end 72 includes a holder 78 for holding at least one epicardial electrode 112, 212 therein. In one embodiment, holder 78 is configured to hold several epicardial electrodes 112, 212, lined up such that each epicardial electrode is proximal to the one ahead of it. In another embodiment, holder 78 is configured to hold one epicardial electrode at a time. In yet another embodiment, several holders 78 are included, each of which can hold one epicardial electrode at a time. Body 76 includes at least one articulating element 80. In a preferred embodiment, multiple articulating elements 80 are included, providing delivery tool 70 with increased flexibility. In one embodiment, body 76 further includes at least one strap 82 for containment of electrical lead 115, 215, and vacuum tube 68. Electrical lead 115, 215 has a standard lead connector 90 for connection to a pacemaker at a proximal end thereof. Vacuum tube 68 has a suction connector 92 for connection to a suction pump at a proximal end thereof. In one embodiment, a vacuum tube control wire 84 is attached to body 76. Vacuum tube control wire 84 is configured to enable vacuum tube 68 to be disconnected from distal end 72 after permanent attachment of epicardial electrode 112, 212. In one embodiment vacuum tube 68 is connected to vacuum valves 66 at distal end 72 by a screw mechanism. Control wire 84 is connected to a safety lock mechanism in distal end 72 that protects vacuum tube 68 from premature disconnecting. Pulling or pushing control wire 84 releases the lock mechanism and enables disconnecting of vacuum tube 68 from distal end 72 by turning or unscrewing. Proximal end 74 includes a handle 73 having control mechanisms for control of movement of delivery tool 70, specifically by controlling articulating elements 80, of vacuum tube 68, and of vacuum tube control wire 84. The delivery system of the present invention enables optimal control of electrode steering, positioning, temporary non-wounding fixation, relocating when necessary, and permanent fixation.

### Positioning and Implantation of CRT System:

Right side component 102 or 202 is positioned within right atrium 16 and right ventricle 18 using methods which are commonly known in the art. Left side component 104 or 204 is positioned on the epicardial surface of the heart. In a preferred embodiment, placement of epicardial electrodes 112 is done by a minimally invasive procedure. In one embodiment, a thoracic sub-xiphoid minimally invasive surgical procedure, routinely used for direct surgical exposure of the pericardial space to allow for epicardial (and other cardiac) manipulations, is used for placement of epicardial electrodes 112. Each of the epicardial electrodes 112 is inserted by a thin steering instrument enabling maximum flexibility in steering each of the electrodes to its desired fixing site.

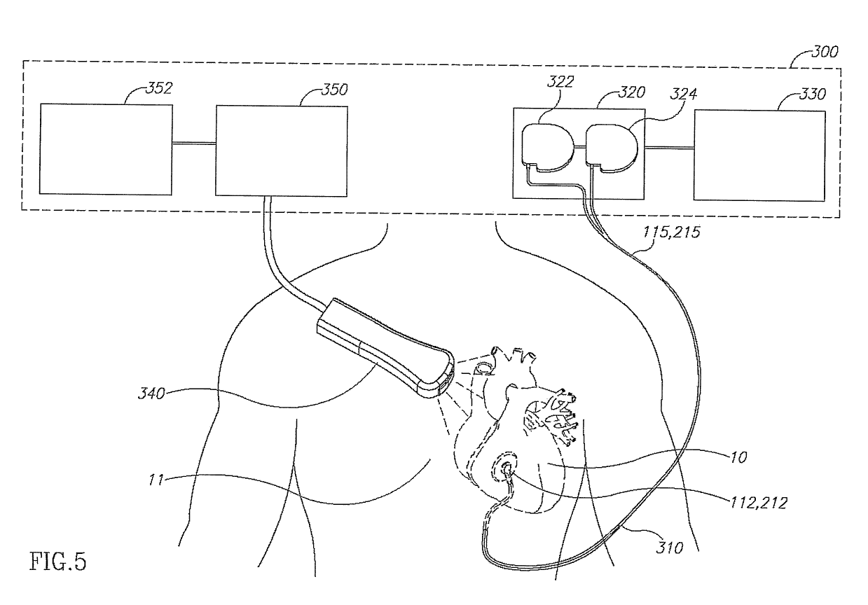

Reference is now made to FIG. 5, which is a schematic illustration of a positioning system 300 for positioning of epicardial electrodes 112 or 212 on the epicardial surface of heart 10. As shown in FIG. 5, heart 10 is positioned within a chest 11 of an individual. Components in FIG. 5 which are positioned internally within the body of the individual are depicted with broken lines, and components in FIG. 5 which are external to the body of the individual are depicted with unbroken lines. Heart 10 is depicted in an internal cavity of the chest wall. Positioning system 300 includes a steering instrument 310 with epicardial electrodes 112, 212 positioned therein, a testing module 320 and a testing monitor 330. In one embodiment, positioning system 300 further includes an external imaging device 340, an imaging processor 350, and an imaging monitor 352.

Steering instrument 310 is a thin, flexible endoscope or catheter, which can be used in a minimally invasive procedure to deliver epicardial electrodes 112 or 212 to heart 10. In one embodiment, steering instrument 310 is an endoscope having telescoping capabilities, and is insertable via a minimally invasive procedure such as a sub-xiphoid thoracic minimally invasive procedure. Epicardial electrodes 112 or 212 with leads 115, 215 are stored within steering instrument 300. Leads 115, 215 are connectable to pulse generator 106 or 207, and are also connectable to testing module 320. Alternatively, wireless communication between epicardial electrodes 112, 212 and either pulse generator 106, 207 or testing module 320 is possible. Testing module 320 includes a sensing component 322 and a pacing component 324, and is configured to sense electrical activation in an area of both intracardiac electrodes 108, 110 and epicardial electrodes 112, 212, and/or to provide electrical stimulation to this area for testing of pacing. In a preferred embodiment, the area of epicardial electrodes 112, 212 is left ventricle 22, but in alternative embodiments may be other areas of heart 10. Furthermore, sensing component and pacing component 324 may be in communication with one another, such that sensing of electrical activation (or lack thereof) can influence whether or not pacing component 324 should provide stimulation or not. In one embodiment, testing module 320 further includes a processor for integrating input and output data. It should be readily apparent that testing module 320 is designed to mimic an implantable pulse generator, and is useful in determining accuracy of placement as well as efficacy of epicardial electrodes 112, 212. Furthermore, testing module 320 can conduct tests and/or simulations during a procedure. Examples of features present in testing module 320 include determinations regarding which of the electrodes should be used for processing of sensing data, which of the electrodes should be used for application of electrical impulses, as well as determinations regarding time, duration and magnitude of pulse signals at each electrode, and any other processing capabilities which can aid in setting up a system for CRT.

External imaging device 340 is a device which can provide imaging of the heart wall, and more particularly, the ventricle wall during a cardiac cycle. In a preferred embodiment, external imaging device 340 is an echocardiogram having Tissue Doppler Imaging (TDI) software, and includes a hand-held component which is positionable on a surface of the chest 11. In one embodiment, external imaging device 340 is completely non-invasive. In an alternative embodiment, imaging of cardiac wall regions can be done separately from or in combination with external imaging device 340. Such alternatives can include, for example, the Biosense Webster (a Johnson & Johnson company) CARTO XP System catheter-based cardiac electrophysiological (EP) mapping or the Biosense Webster (a Johnson & Johnson company) Cordis Webster NAVI-STAR catheters which are indicated for electrophysiological and electromechanical mapping of cardiac structures. In alternative embodiments, an imaging mechanism is included on steering instrument 310, and can be used separately from or in combination with external imaging device 340. External imaging device 340 is in communication with imaging processor 350. Imaging processor 350 processes signals received from imaging device 340, and presents processed results as an image via imaging monitor 352. Images can be presented to the user in two or three dimensions, or possibly with color coding. Information viewed on imaging monitor 350 helps in determining abnormal conduction/contraction areas, and accordingly optimal sites for epicardial electrode 112 placement, and correct positioning of epicardial electrodes 112, 212. Specifically, such information may include contraction times or velocities of various locations of the ventricle wall during a cardiac cycle, providing high resolution information on regional wall motion dynamics for use in determination of positioning of epicardial electrodes 112, 212.

TDI can demonstrate unique patterns of activity in individual patients. Thus, it is possible to map areas of delayed contraction time or slow contraction velocity, which are generally due to conduction disturbances. By specifying locations of late contraction using TDI or alternative techniques, it is then possible to place epicardial electrodes 112 directly on the specified locations in order to optimize pacing. It should be noted that one or several locations may be identified and used for placement of epicardial electrodes. Both the number and the locations of the sites for epicardial electrode placement are determined by the imaging technique. In one embodiment, TDI is done prior to implantation. In another embodiment, TDI is performed simultaneously, and is used for on-line monitoring of the implantation procedure.

In addition to specifying locations for epicardial electrode placement, TDI or other imaging techniques can be used for safety purposes as well. Specifically, some of the largest blood vessels of the heart are located on the epicardium, such as, for example, the anterior descending and circumflex arteries and their branches, and the great cardiac vein and its branches. To avoid puncture of these blood vessels during electrode active fixation, veins and arteries may be injected with radiopaque contrast media. Invasive components of system 100 or 200 (such as, electrodes or steering instruments) can be marked by radiopaque materials, or by other markers which would make them visible in an imaging procedure. In alternative embodiments, epicardial electrodes 112, 212 further include Doppler transducers or sensors capable of detecting blood flow in vessels, thus allowing avoidance of these vessels during electrode fixation to the epicardium.

In alternative embodiments, epicardial electrodes 112 are attached to other areas of the heart. For example, in some cases it might be advantageous to include epicardial electrodes on the epicardium of the right side of the heart, such as the right ventricle or right atrium.

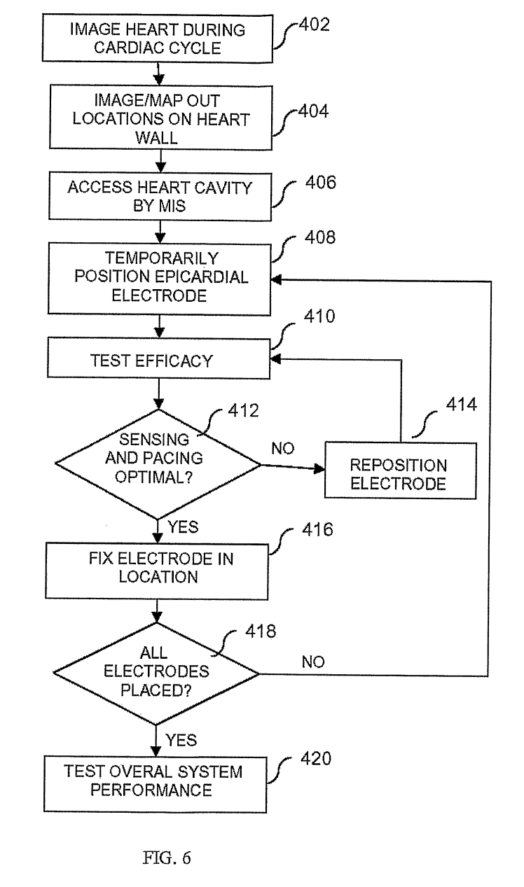

Prior to inserting epicardial electrodes 112, 212, right atrium lead 108 and right ventricle lead 110 are inserted transvenously into right atrium and ventricle, respectively. Reference is now made to FIG. 6, which is a flow chart diagram of a method of positioning of epicardial electrodes 112, 212 in accordance with a preferred embodiment of the present invention. The method of FIG. 6 is performed using system 300 depicted in FIG. 5. First, imaging device 340 images (step 402) the heart during a normal cardiac cycle. Imaging processor 350 images or maps out (step 404) locations on the heart wall based on imaging information received from imaging device 340, and displays these locations on imaging monitor 352. Specifically, locations of late contraction and/or poor velocity are identified and noted. Merging of electrical and anatomical information into a 3-dimensional representation of the heart delineating areas of interest can be done using processing techniques and/or software. Next, the user accesses (step 406) the heart cavity in a minimally invasive surgical procedure (MIS), such as a sub-xiphoid procedure, using steering instrument 310. The user then temporarily positions (step 408) a first epicardial electrode 112 or 212 on the heart surface at a location identified by imaging device 340. This temporary attachment is accomplished by vacuum or other non-damaging means. The user then attaches the lead of the first epicardial electrode to testing module 320. Testing module 320 tests (step 410) the efficacy of the epicardial electrode in that particular position. Specifically, testing includes evaluating the ability to sense the cardiac intrinsic electrical activity and to pace the underlying epicardial wall (without pacing other internal parts of the body such as the diaphragm, for example). In one embodiment, imaging device 340 images the heart during the minimally invasive procedure, either instead of or in addition to imaging prior to the procedure. In this way, the user can test the efficacy (step 410) of the epicardial electrode while actively changing the positioning and location of the electrode. If positioning is optimized (decision step 412), the user fixes (step 416) the electrode in place by, for example, a penetrating screw. If not, the user repositions (step 414) the electrode and retests (step 410) it. This procedure is repeated for any number of electrodes. In one embodiment, a different steering instrument 310 is used for each individual electrode. In an alternative embodiment, all epicardial electrodes share the same steering instrument 310. In this embodiment, the electrodes are set in steering instrument 310 in order of insertion, and can be used by guiding electrodes one after the other to their position. In either embodiment, when the desired number of electrodes are in place (decision step 418), a testing module tests (step 420) the overall performance of the system.

Once the testing phase is complete, the user disconnects the leads from testing module 320 and connects them to a pulse generator implanted within the chest wall. In one embodiment, the pulse generator is the same pulse generator 106 that the right side component is in communication with. In this embodiment, pulse generator 106 is implanted in a routine procedure by placement under the skin of the upper chest (generally on the left side). In this embodiment, epicardial electrode leads 112 are tunneled under the frontal chest skin to the location of pulse generator 106. In an alternative embodiment, the pulse generator connected to epicardial electrodes 212 is a separate pulse generator 207. In this embodiment, right side component 202 is attached to a first pulse generator 206, implanted in a routine procedure and placed under the skin of the upper chest. Left side component 204 is connected to a second pulse generator 207 which is implanted in a different location, such as under the frontal skin of the upper abdomen.

Synchronization of first and second pulse generators 206 and 207 can be accomplished in several ways. In a first embodiment, at least one of epicardial electrodes 212 is used as a sensing electrode and is implanted over a key spot to enable relatively early sensing of the intrinsic cardiac activation during a cardiac cycle. Based on this sensing, second pulse generator 207 sets the timing of epicardial electrode 212 activation. In an alternative embodiment, a telemetry mechanism is set up between the two individual pulse generators 206 and 207 to enable synchronization. The telemetry module in both pulse generators 206 and 207 enable delivery of data received and processed by one pulse generator to be communicated to the other pulse generator. This allows the two separate pulse generators 206 and 207 to act in a reciprocatory manner, and as one unit.

In one embodiment, the separate left side component 204 and the second pulse generator 207 can be added to a typical CRT system already present in an individual, to help correct unsuccessful transvenous left side synchronization. In other embodiment this setup could be advantageous for patients with a standard dual chamber pacemaker of right atrium and right ventricle transvenous leads who later develop cardiac asynchrony. The use of a separate pulse generator 207 would make this type of addition possible since the standard pacemaker would not support additional leads. This setup may also be useful for patients in whom tunneling the left side leads from the area of the sub-xiphoid to the upper chest is unattainable due to other medical circumstances.

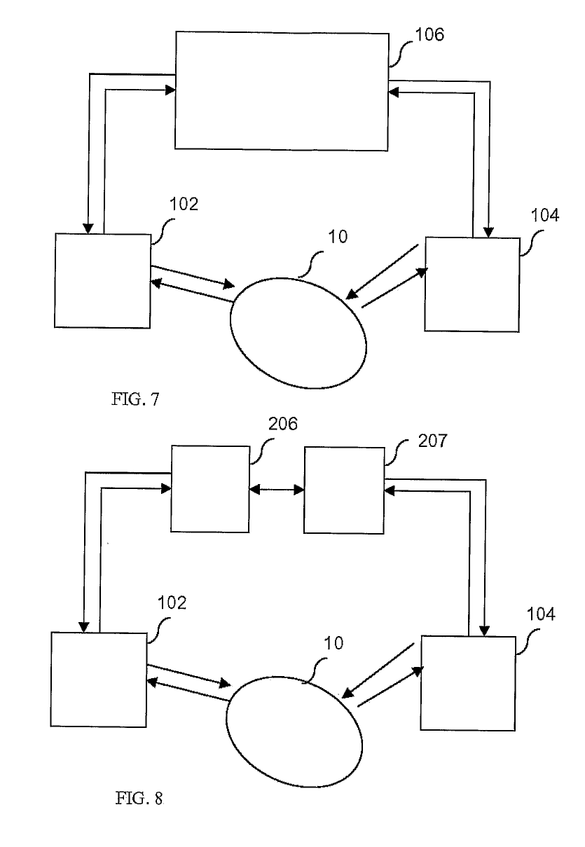

Reference is now made to FIG. 7, which is a schematic illustration of an overview of a method of pacing using the system of the present invention. In one embodiment, right side component 102 is able to sense electrical activity from heart 10 and to pace left and right sides by pulses sent by pulse generator 106. In a preferred embodiment, right side component 102 and left side component 104 are both able to sense electrical activity from heart 10, and to pace the underlying tissue by pulses sent from pulse generator 106. Arrows are shown in both directions to indicate the dual function (i.e., sensing and pacing) of right and left side components 102, 104. Electrical data representing electrical activity in the myocardium is sensed by right and left side components 102, 104, and sent to pulse generator 106. This electrical activity is continuously monitored and collected. Pulse generator 106 calculates performance parameters from the raw data input from all or part of both internal and epicardial electrodes, and constructs a pacing scheme to optimally synchronize the cardiac chambers to yield optimal cardiac output. Pacing signals are sent from pulse generator 106 to right and left side components 102, 104.

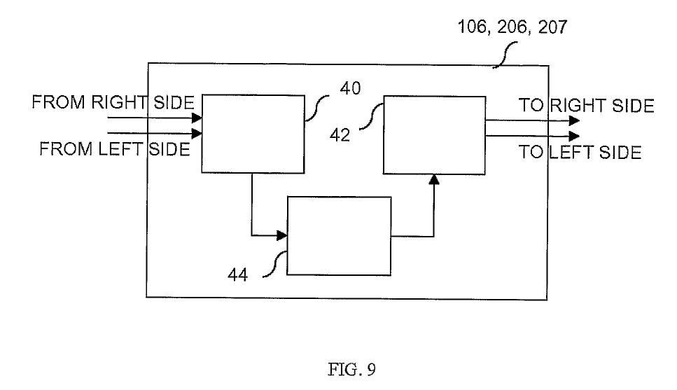

Reference is now made to FIG. 8, which is a schematic illustration of an overview of an alternative method of pacing using the system of the present invention. In one embodiment, right side component 202 is able to sense electrical activity from heart 10 and to pace left and right sides by pulses sent by pulse generators 206, 207. In a preferred embodiment, right side component 202 and left side component 204 are both able to sense electrical activity from heart 10, and to pace the underlying tissue by pulses sent from individual pulse generators 206 and 207. Arrows are shown in both directions to indicate the dual function (i.e., sensing and pacing) of right and left side components 202, 204. Electrical data representing electrical activity in the myocardium is sensed by right and left side components 202, 204, and sent to pulse generators 206 and 207, respectively. This electrical activity is continuously monitored and collected. Pulse generators 206 and 207 communicate with one another by telemetric means. Each of pulse generators 206 and 207 calculates performance parameters from the raw data input, and shares information with one another. Each of pulse generators 206 and 207 processes both its own collected data and the data received from the other pulse generator, and based on the processed data, each of pulse generators 206 and 207 constructs a pacing scheme to optimally synchronize the cardiac chambers to yield optimal cardiac output. Pacing signals are sent from pulse generator 206 to right side component 202 and from pulse generator 207 to left side component 204.

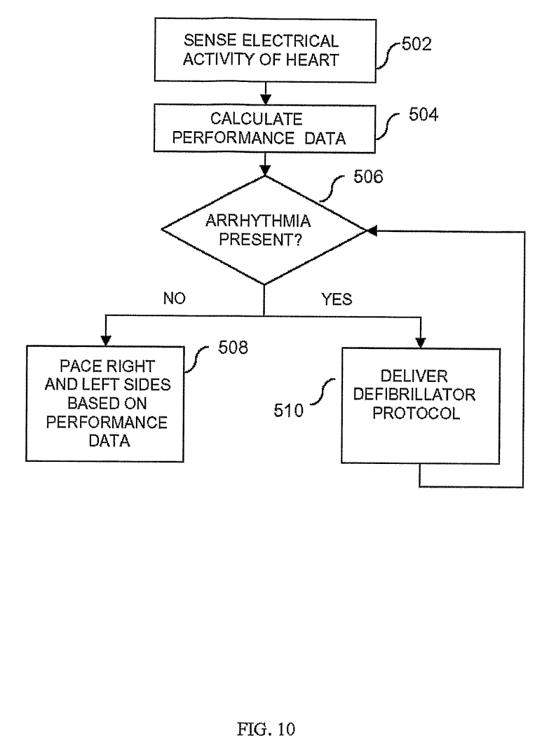

Reference is now made to FIG. 9, which is a block diagram illustration of the components of pulse generator 106, 206 or 207. Pulse generator 106, 206, 207 has an input module 40, an output module 42, and a processor 44. Input module 40 receives raw signals from electrical activity from the right and left sides of heart 10, from all or part of the electrodes 108, 110, 112. Raw signals are sent to processor 44, which calculates performance parameters, and constructs pacing schemes. This information is sent to output module 42, which sends pacing signals in accordance with determined procedures to right side component 102 or 202 and to left side component 104 or 204.

Electrical signals received by input module 40 of the pulse generator can be processed by various methods known in the art. In one embodiment, the processing of data is as follows. Each electrode waveform is digitally filtered to remove unwanted noise, and timing of specific cardiac events is calculated. One way to accurately time cardiac events is by calculating the first derivative of the filtered signal. Timed cardiac events include, for example:
1. Local activation time (LAT). LAT at each electrode is determined by calculating the minimum of the first derivative plot (corresponding to dF/dtmin where F is the filtered signal). This method is in accordance with the method of Spach and Dolber, (“Relating extracellular potentials and their derivatives to anisotropic propagation at a microscopic level in human cardiac muscle: Evidence for electrical uncoupling of side-to-side fiber connections with increasing age” Circ Res. 1986; 58:356-371) who related theoretically and experimentally cardiac extracellular waveforms and transmembrane potentials to each other in a propagation at a microscopic size scale (<200 μm). The study demonstrated that the maximum transmembrane first derivative (Vmax) and the minimum first derivative of the extracellular unipolar recording would occur simultaneously, i.e. that the negative peak of the first derivative of the extracellular potential would provide a marker for the instant maximum rate of increase of the cellular depolarizing current during excitation.
2. Activation duration (QRS complex duration).
3. Cycle length, determined as R-R (also corresponding to LAT-LAT) intervals.
   Events other than the ones listed above may be measured as well.

The measured parameters are calculated for individual electrodes separately.

These calculations are then integrated to provide an activation sequence and pattern, conduction velocity and heart rate variability. By determining LAT at each electrode, a 3-dimensional figure of the activation sequence and conduction velocity can be calculated. This information is used for constructing a pacing scheme. For example, a larger than normal activation delay between the right atrium, sensed by electrode 108, and the left ventricle, sensed by one of epicardial electrodes 112 can be corrected by applying early activation pulses by other epicardial electrode(s) 112.

Defibrillator Option:

In certain cases, defibrillation capabilities are desired, particularly since approximately one-sixth of patients who receive CRT treatment are believed to be at high risk of sudden cardiac arrest. The unique setup of both intracardiac right side electrodes 108, 110 and the epicardial electrodes 112, 212 of the present invention may enable more efficient Anti Tachycardia Pacing (ATP) treatment than current systems, and is expected to further reduce the need for using painful defibrillator shocks to terminate arrhythmias in these high-risk patients.

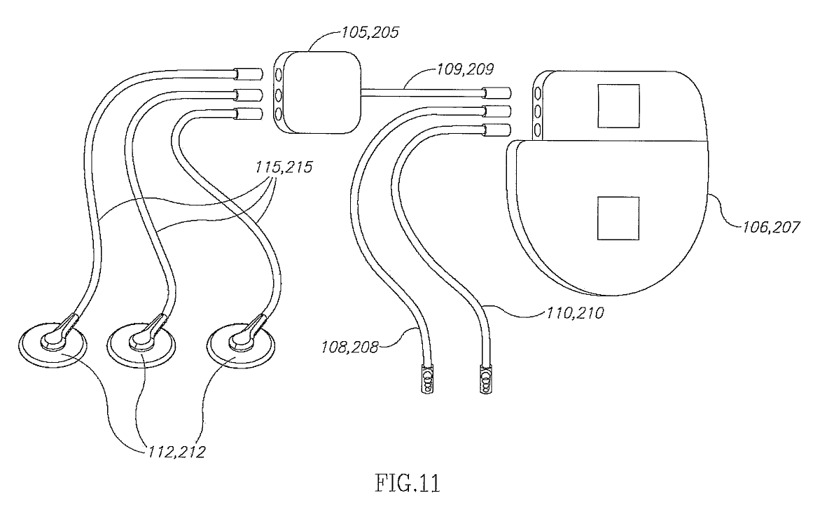

Reference is now made to FIG. 10, which is a flow chart illustration of a method of pacing with a defibrillator option, in accordance with one embodiment of the present invention. Left and right side components 102/202 and 104/204 sense (step 502) electrical activity of the heart. Signals from the sensed activity are then sent to pulse generator 106 or pulse generators 206 and 207. Processors 44 of the pulse generator calculate (step 504) performance data, and determine whether an arrhythmia is present (decision step 506). If there is no arrhythmia present, signals are sent from output module 42 to pace (step 508) the right and left sides of the heart based on calculated measures and performance data as described above. If an arrhythmia is detected, output module 42 delivers (step 510) a defibrillator protocol to epicardial electrodes 112, 212, and to right atrium and ventricle leads 108 and 110.

The defibrillator protocol includes best-fit pacing protocols (PP) to terminate tachyarrhythmias and restore normal activity. In response to abnormal electrical activity, the pulse generator characterizes the nature of the arrhythmia (spatially and temporally), and runs a selected ATP protocol or delivers defibrillator shock. Due to the high coverage of the electrical activity by its electrode array, the system of the present invention can better determine the nature of the arrhythmia, and can manipulate the arrhythmia by conducting a more sophisticated ATP protocol by pacing from part or all the electrodes. This higher sensing/pacing ability of the system may allow for treating of arrhythmias in a “softer” manner as compared to current devices.

In additional embodiments, the delivery system of the present invention can also be used for other manipulations which require high steering capability in the pericardium cavity. For example, steering instrument 310 can be used to steer an ablation catheter to a desired location on the epicardium or for a device for occlusion of the left atrial appendage, which are both procedures known in the art of cardiac therapy.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for cardiac resynchronization therapy of a heart, the system comprising:
a right side component including at least one right side lead, said right side lead positionable inside the heart and configured to provide sensing signals from sensed electrical activity of the heart and to receive pacing signals for pacing electrical activity of the heart;
a left side component comprising at least one epicardial electrode, said electrode positionable on an epicardial surface of the heart and configured to provide sensing signals from sensed electrical activity of the heart and to receive pacing signals for pacing electrical activity of the heart; and
at least one pulse generator for receiving from said right side component and said left side component said sensing signals provided from sensed electrical activity and for sending said pacing signals to said right side component and said left side component.

2. The system of claim 1, wherein said at least one right side lead comprises a right side atrium lead and a right side ventricle lead.

3. The system of claim 1, wherein said left side component comprises multiple epicardial electrodes.

4. The system of claim 1, wherein said at least one pulse generator comprises: an input module, an output module, and a processor.

5. The system of claim 4, wherein said processor comprises a calculator for calculating performance parameters from said sensing signals and for constructing a pacing procedure.

6. The system of claim 4, wherein said processor comprises a defibrillator for providing defibrillation signals to said right side component and said left side component.

7. The system of claim 1, wherein said at least one pulse generator is implantable in a chest cavity by a minimally invasive procedure.

8. The system of claim 1, further comprising a steering catheter for positioning of said epicardial electrode on said epicardial surface of the heart.

9. The system of claim 1, further comprising an imaging device for determining at least one location having a conduction disturbance.

10. The system of claim 9, wherein said imaging device is non-invasive.

11. The system of claim 9, wherein said at least one epicardial electrode is positioned at said at least one location.

12. A kit for providing enhanced cardiac resynchronization therapy to an existing single side pacing system or double side cardiac pacing system, the existing system having at least a right side component and a first pulse generator in communication with at least the right side component, the kit comprising:
a left side component comprising at least one epicardial electrode, said electrode positionable on an epicardial surface of the heart and configured to provide sensing signals from sensed electrical activity of the heart and to receive pacing signals for pacing electrical activity of the heart;
a second pulse generator for receiving from said left side component said sensing signals provided from sensed electrical activity and for sending said pacing signals to said left side component; and
a telemetry module in communication with said second pulse generator, for sending sensing signals and pacing signals to the first pulse generator.

13. The kit of claim 12, wherein said left side component comprises multiple epicardial electrodes.

14. The kit of claim 12, wherein said second pulse generator comprises: an input module, an output module, and a processor.

15. The kit of claim 14, wherein said processor comprises a calculator for calculating performance parameters from said sensing signals and for constructing a pacing procedure.

16. The kit of claim 14, wherein said processor comprises a defibrillator for providing defibrillation signals to said left side component.

17. The kit of claim 12, wherein said second pulse generator is implantable in an abdominal cavity by a minimally invasive procedure.

18. The kit of claim 12, further comprising a steering catheter for positioning of said epicardial electrode on said epicardial surface of the heart.

19. The kit of claim 12, further comprising an imaging device for determining at least one location having a conduction disturbance.

20. The system of claim 19, wherein said imaging device is non-invasive.

21. The system of claim 19, wherein said at least one epicardial electrode is positioned at said at least one location.

22. A method for positioning a system for cardiac resynchronization therapy, the method comprising:
imaging a cardiac region of the body during a regular cardiac cycle;
determining at least one location having a conduction disturbance based on said imaging;
inserting a steering instrument into the cardiac region, the steering instrument having at least one epicardial electrode stored therein;
temporarily placing said at least one epicardial electrode on an epicardial surface at said at least one location;
testing said at least one epicardial electrode for efficacy;
determining a placement location based on said imaging and said testing; and
fixing the at least one epicardial electrode to an epicardial surface at said placement location.

23. The method of claim 22, further comprising repeating said steps of temporarily placing, testing, determining and fixing for a second epicardial electrode.

24. The method of claim 22, further comprising pacing said at least one epicardial electrode.

25. The method of claim 22, further comprising defibrillating said at least one epicardial electrode.

26. The method of claim 22, wherein said imaging is done by Tissue Doppler Imaging.

27. The method of claim 22, wherein said inserting is done by a minimally invasive procedure.

28. The method of claim 27, wherein said minimally invasive procedure is a sub-xiphoid procedure.

29. The method of claim 22, wherein said temporarily placing is done by vacuum.

30. The method of claim 22, wherein said testing is done by a testing module.

31. The method of claim 22, wherein said testing is done by further imaging.

32. An epicardial electrode for temporary attachment to an epicardium wall, said electrode comprising:
a contact portion configured to be in electrical contact with an epicardium of a heart; and
a suction portion, said suction portion comprising:
a flexible band surrounding said contact portion;
at least one suction valve on said contact portion; and
a vacuum tube connecting said at least one suction valve to a suction pump.

33. The epicardial electrode of claim 32, wherein said contact portion further comprises a fixation element for permanent fixation of said epicardial electrode to an epicardium wall.

34. The epicardial electrode of claim 32, wherein said at least one suction valve comprises multiple suction valves.

35. A delivery tool for minimally invasive placement of epicardial electrodes, the tool comprising:
a distal end having a holder for holding of at least one of the epicardial electrodes;
a proximal end having a handle; and
a body connecting said distal and proximal ends, wherein said body includes at least one articulating element,
wherein said handle includes controls for controlling said at least one articulating element and said at least one epicardial electrode.

36. The delivery tool of claim 35, wherein said body includes multiple articulating elements.
